(19) United States
(12) Patent Application Publication
(10) Pub. No.: US 2008/0311595 A1
(43) Pub. Date: Dec. 18, 2008

(54) Rapid fungal detection assay and product

(76) Inventor: Inger Mattsby-Baltzer, Goteborg (SE)

Correspondence Address: LYNN E BARBER, P O BOX 16528, FORT WORTH, TX, 76162, US

(21) Appl. No.: 12/151,680

(22) Filed: May 8, 2008

Related U.S. Application Data
(60) Provisional application No. 60/936,133, filed on Jun. 18, 2007.

(51) Int. Cl. G01N 33/53; C12M 1/00

(52) U.S. Cl. 435/7.31; 435/287.1; 435/287.2

(57) ABSTRACT

The present invention relates to the use of an assay and product for rapid detection of fungi in a variety of samples suspected of containing fungi, particularly swab samples from the human vaginal area, but also in other environments such as in food and feeds. The product of the invention is suitable for use in a variety of different environments, including in clinical settings, at the point of care, and by patients themselves (bedside kit).

### CROSS-REFERENCE TO RELATED APPLICATIONS

This application claims priority from U.S. Provisional Application Serial No. 60/936,133 filed Jun. 18, 2007.

### BACKGROUND OF THE INVENTION

1. Field of the Invention

The present invention relates to an assay and product for rapid detection of fungi in a variety of samples suspected of containing fungi, for example the yeast, Candida, particularly swab samples from the human vaginal area. The product of the invention is suitable for use in a variety of different environments, including in clinical settings, at the point of care, and by patients themselves (bedside kit).

2. Description of the Related Art

Fungal infections and growth may occur at many places including the human or animal body, e.g. in the vagina or in the oral cavity. Invasive fungal infections are increasing because of the growing number of immunocompromised patients (Jones, J. M. 1990. Laboratory diagnosis of invasive (systemic) candidiasis. Clin Microbiol Rev 3:32-45). Many of these infections occur in critically ill patients suffering from an underlying disease. Over the past decades “Candida overgrowth” has increasingly been a problem as a result of several factors including the abundant use of antibiotics in medicine and agriculture.

Candida vaginitis is an infection of the vagina. It causes a foul smelling, sticky, white-yellow discharge that may be accompanied by itching, burning and swelling. Such an infection can also make walking, urinating, or intercourse painful. Generally, the vaginal cavity exhibits an aqueous environment containing secreting glands whose fluids create an acidic pH in the range of about 4-5. In its normal state, the lining of the vagina secretes a fluid that is fermented to an acid by bacteria that are normally present. Many women harbor the yeast C. albicans in the vagina but most have no symptoms and it is harmless to them. This acidity is a protective mechanism that helps to protect the vagina from invasion by other microorganisms.

Certain drug therapies can alter the balance of natural microorganisms that are present in the vagina, and thereby promote the growth of Candida. Examples include the extended use of antibiotics, steroids and oral contraceptives with high estrogen content. Other factors that may cause Candida vaginitis include diabetes, pregnancy, using antihistamines, iron, folate, vitamin B12, or zinc deficiency. Factors that may weaken the immune system can also cause candidiasis. Tight fitting pants and the reactions to chemical ingredients found in soaps and detergents may also lead to Candida vaginitis. Treatment with topical antifungal compositions, such as creams or suppositories, is normally the first choice of treatment for mild to moderate yeast infections. Serious infections, however, require a longer course of treatment.

Because anti-Candida antibodies are often present in healthy individuals, which is considered to be a consequence of immunization from Candida in the commensal flora, antibody tests are not a recommended method of detecting Candida (Odds, F. C., and E. G. Evans. 1980. Distribution of pathogenic yeasts and humoral antibodies to Candida among hospital in patients. J Clin Pathol 33:750-6). An antibody test that relates to a method of diagnosing Candida infection using mannan depleted antigen detecting antibodies is described in U.S. Pat. No. 6,916,626 B1.

The immunoassay technique, which relies on the specific binding action between an antigen or a hapten and a corresponding antibody, has proven to be a reliable method for determining the presence (or absence) of a pathogen in a specimen. A class of devices known as immunochromatographic test (ICT) devices uses the immunoassay technique in combination with a label that is conjugated with the antibody and is now commonly used for rapid, reliable field tests to determine the presence or absence of a particular analyte. The label, when attached to antibody/antigen molecules that are then amassed together in a specific, restricted area, becomes readily detectable by the naked human eye, or by a scanning device, depending on the type of label used. In general, the label can be a particle of latex, gold, or carbon, a radioactive particle, a magnetic particle, or have other physical or chemical properties that allow it to be fixed or attracted to a certain defined area. ICT devices that use the sandwich technique are particularly easy to use. With this technique, labeled antibody that binds with the specific antigen to be assayed is mixed with the sample that is suspected of containing the specific antigen. If the antigen is present in the sample, the labeled antibody binds with the antigen to form a label-antibody-antigen complex. A second antibody that is immovably fixed at a test zone and that also binds with the specific antigen binds the label-antibody-antigen complex at the test zone. A positive result is made visible by the accumulation of the label at the test zone. Such devices are economical and can be used by unskilled workers.

Several types of such ICT devices are known. Most are the “dipstick” type in which a test strip is encased in a hollow housing with a bibulous pad extending from one end. This pad is dipped into the liquid sample and draws the liquid by capillary (“wicking”) action up onto a section of the test strip that contains a labeled antibody, i.e., a label conjugated to an antibody that will specifically bind with the antigen being assayed. The labeled antibody moves with the liquid that is being drawn by the capillary action further along the test strip and, if the specific antigen to which the antibody binds is present in the liquid sample, the labeled antibody will bind with the antigen, forming a labeled antibody-antigen complex. This complex continues to flow with the liquid along the test strip. Downstream from the area containing the conjugated antibody is a test zone. This test zone is typically a nitrocellulose pad into which a second binding partner, an antibody that binds to a second epitope of the same antigen as the labeled antibody, has been immovably fixed. The fixed antibody will attach to the labeled antibody-antigen complex that flows onto the test zone and will bind the complex to the test zone. The presence of an antigen being assayed is then visible as a stripe across the test zone or is otherwise readily detectable. The excess liquid continues to flow past the test zone across a control zone. There are a number of well-known means in the field of immunoassay of creating a control zone, such as embedding into the control zone a binding partner that binds non-specifically to one or more of the labeled antibodies contained on the conjugate section, or to a labeled analyte added to the liquid sample for the purpose of binding with the non-specific binding partner at the control zone. A properly completed test will always show a visible stripe across the control zone or, if a radioisotope or magnetic particle is used as a label, an otherwise readily detectable stripe. Typically, for those devices using a colored label, the housing of the ICT device has a window through which to view the test zone and the control zone. Devices of this type have been disclosed in May et al. (U.S. Pat. No. 5,622,871; issued Apr. 22, 1997) and Charlton et al. (U.S. Pat. No. 5,714,389; issued Feb. 3, 1998).

Chandler (U.S. Pat. No. 5,869,345; issued 1999, U.S. Pat. No. 5,877,028; issued 1999) discloses an ICT device that is a two-panel card containing a test strip on one panel and a sample well on the other panel. The Chandler device also uses the sandwich technique described above with the dipstick devices, but has a particular advantage in that it allows the sample to be prepared for the test directly on the test card, rather than in a separate vessel. When the test card is closed, liquid from the sample well flows onto the test strip. As with the other devices, a window is provided through which to view the results.

Immunoassays are the established procedures for the diagnosis of Candida. They can be designed to either detect Candida antigens, or host antibodies reactive against Candida antigens. Several immunoassays are commercially available for the detection of Candida antigens in sera or other body fluids. However, these assays lack either sensitivity or specificity or both. There also are not any rapid lateral flow immunoassays for detection of Candida developed commercially nor are any described in any patents.

A method using a rapid immunochromatographic test for determining and diagnosing inflammatory enteric disease is described in U.S. Pat. No. 6,727,073 B1. The method tests for the presence of at least one enteric pathogen and at least one of certain inflammatory enteric disease markers.

Rapid lateral flow immunoassays have been commercially developed, for example: QuickVue®Chlamydia test available from Quidel. A labeling complex comprising antibodies specific for an epitope on the lipopolysaccharide antigen of Chlamydia is present within the labeling zone. Immobilized antibody specific for the same or another epitope of the lipopolysaccharide antigen of Chlamydia is located in the capture zone.

Another commercially developed test is NOW® Streptococcus pneumoniae Antigen Test available from Binax Inc. It is an in vitro rapid immunochromatographic assay for the detection of S. pneumoniae antigen in the urine of patients with pneumonia and in the cerebral spinal fluid (CSF) of patients with meningitis.

In Candida species which are the most common fungi isolated from patients with vaginitis, the yeast cells are surrounded by a rough, rigid cell wall that represents 20-25% of the dry weight of the cells (Klis, F. M. 1994. Review: cell wall assembly in yeast. Yeast 10:851-69.). The cell wall of C. albicans and Saccharomyces cerevisiae consists of about 85-90% polysaccharide, 10-15% protein, and a small amount of lipids (Nelson, R. D., N. Shibata, R. P. Podzorski, and M. 5 J. Herron. 1991. Candida mannan: chemistry, suppression of cell-mediated immunity, and possible mechanisms of action. Clin Microbiol Rev 4:1-19. Nguyen, T. H., G. H. Fleet, and P. L. Rogers. 1998. Composition of the cell walls of several yeast species. Appl Microbiol Biotechnol 50:206-12). The polysaccharide components consist of mannan, glucan, and a small amount of chitin. Most of the proteins are covalently linked to the mannan (mannoprotein), which is located in the outermost layer of the cell wall. A fraction of the proteins is also covalently linked to glucan (Kapteyn, J. C., L. L. Hoyer, J. E. Hecht, W. H. Muller, A. Andel, A. J. Verkleij, M. Makarow, H. Van i Den Ende, and F. M. Klis. 2000. The cell wall architecture of C. albicans wild-type cells and cell wall-defective mutants. Mol Microbiol 35:601-11). The proportions of these different components vary with the yeast species.

The glucan microfibriles are located mostly in the inner part of the cell wall. β(1-3)-glucans are unique for all medically important fungi and are shed during growth (Miyazaki, T., S. Kohno, K. Mitsutake, S. Maesaki, K. Tanaka, and K. Hara. 1995. Thus, determination of β(1-3)-glucans appear to be a useful marker in the laboratory diagnosis of fungal infections such as Candida vaginitis.

One analysis method of β (1-3)-glucans is based on the binding of the polysaccharide to a coagulation factor, factor G, present in blood cell lysate from the Limulus horse shoe crab. This glucan test, however, has some limitations. It does not react exclusively with β (1-3) glucans, since also (1 3)(1-4)-a-D-glucan (negaran) and (1-2)(1-3)(1-6)-a-D 10 glucan (yeast a-D-mannan), and (1-6)-D-glucan (gyrophoran) may activate the G factor (Adachi, Y., N. Ohno, T. Yadomae, Y. Suzuki, M. Ohsawa, and S. Oikawa. 1990. Thermal denaturation of 5 1-3-beta-D glucans in neutral aqueous solution above 130 degree effect on physicochemical proper ties. Carbohydr Res 198:111-122; Kapteyn, J. C., L. L. Hoyer, J. E. Hecht, W. H. Muller, A. Andel, A. J. Verkleij, M. Makarow, H. Van i Den Ende, and F. M. Klis. 2000. The cell wall architecture of Candida albicans wild-type cells and cell wall-defective mutants. Mol Microbiol 35:601-11). The reactivity of factor G is also dependent on the molecular weight, conformation and degree of branching of the glucans (Nagi, N., N. Ohno, Y. Adachi, J. Aketagawa, H. Ta 35 mura, Y. Shibata, S. Tanaka, and T. Yadomae. 1993. Application of limulus test (G pathway) for the detection of different conformers of (1->3)-beta-D glucans. Biol Pharm Bull 16:822-8).

Compounds with a high binding specificity for β (1-3)-glucans would be useful tools for providing an analysis of β (1-3)-glucans in any body fluid, such as blood, urine, or in biopsy specimens of patients with suspected fungal infections, and consequently for providing a laboratory diagnosis of fungal infection

Patent WO2004036222 A1 which is made by and assigned to the inventor of this invention discloses monoclonal antibodies reactive with β(1-3) -glucans. More precisely, two monoclonal antibodies, B3B and A10A, reactive with β (1-3)-glucan and/or β(1-3) (1-6)-glucan associated epitopes in free, non-associated form, and/or in cell wall fragments of Candida and Cryptococcus is disclosed. Further, A10A is also reactive with a β(1-6) (1-3)-glucan epitope present on the intact cell surface of C. albicans, Candida parapsilosis, Candida krusei, Candida glabrata and/or C. neoformans.

The advantage of the assay of this invention compared to other Candida tests is that the monoclonal antibodies that are used, A1OA and B3B, are reactive to β(1-3)-glucan which is a unique cell wall compound in all medically important fungi. Furthermore, both antibodies bind to epitopes on the β(1-3)-glucan, which usually is embedded in the cell wall. In addition, A10A is unique in its capacity to recognize an epitope which appears on the surface of the intact yeast cells of Candida species. The advantage of using the deep-seated β(1-3)-glucan as target for the antibody is that the levels of natural antibodies against it are much less as compared to mannoproteins, and thus less interference could be expected in the assay.

Further, A1OA and B3B are reactive with β(1-3)-glucans as present in free form and in cell wall fragments, and in intact fungal cells. It is known that Candida also releases β(1-3)-glucan during growth. A1OA and B3B could be used in combination (as well as separately).

Accurate and rapid identification of C. albicans in vaginal samples of patients suspected of having vaginal candidosis is important to provide appropriate treatment and to minimize the risk of developing resistance to the treatment substances.

For these reasons, it is desirable to provide assays and devices for the fast detection of fungi, for example Candida, in patient samples as well as fungi in other samples such as in foods and feeds. The assays should be sensitive and specific for fungi and should require as few steps as possible for their performance. The assays should further provide rapid results, and should be suitable for use with a variety of samples suspected of containing fungi, for example swab samples for detecting Candida in the vaginal area. The assays should still further be suitable for use in a variety of different environments, including in clinical and other laboratories, at the point of care, and by patients themselves (bedside kit), and for other samples as for example in foods and feeds, in the field or in a plant.

### SUMMARY OF THE INVENTION

The present invention relates to the field of an immunochromatographic lateral flow assay system for the detection of a deep-seated β(1-3)-glucan fungal antigen in extracts from, for example, vaginal swab samples. More particularly, the present invention relates to a diagnostic rapid test, without cultivation, for the diagnosis of fungal infections comprising the monoclonal antibody B3B and A1OA disclosed in patent application WO2004036222A1. The advantage of using the deep-seated β(1-3)-glucan as target for the antibody is that the levels of natural antibodies against it is much less as compared to mannoproteins, and thus less interference could be expected in the assay, making it specially suitable for a quick-test.

An object of this invention is to provide a method for rapidly screening patients for Candida vaginitis that can be performed reliably by unskilled persons which leads to a more cost-effective use of personnel resources than the use of a method that requires the attention of skilled personnel.

Another object of this invention is to provide a quick-test for fungi detection in food, feeds and other products.

### BRIEF DESCRIPTION OF THE DRAWINGS

FIG. 1. The device is constructed of four zones (pads), the sample receiving zone, the labeling and capture (immobilization) zones, and an absorbent zone. In FIG. 1 are shown: sample receiving pad (1), labeling pad (2), immobilization pad (3), immobilization zone (4), control zone (5), absorbent pad (7), back (6).

FIG. 2. A cassette is constructed to contain the four types of pads and an application well. Furthermore the cassette contains a view window for reading. In FIG. 2 are shown: assay matrix (8), back (6), cover (9), sample application port (10), viewing window (11), and air outlet (12).

FIG. 3. A sample collection device. In FIG. 3 are shown: cotton swab (13), bottle (14), screw cap (15), and snap off tip (16).

### DETAILED DESCRIPTION OF THE INVENTION AND PREFERRED EMBODIMENTS THEREOF

Generally, the invention herein is a device for detecting the presence of a fungus in a patient sample, comprising a matrix defining a flow path comprising a labeling zone and a capture zone. Preferably, there is a labeling complex present within the labeling zone that comprises a visible label bound to a labeled binding substance, such as an antibody specific for the fungal β(1-3)-glucan antigen, and a capture binding substance, such as an antibody immobilized within the capture zone that is specific for β(1-3)-glucan fungal antigen. The presence of the fungus may thus be detected by the presence of a fungal antigen, for example, the fungal antigen from Candida albicans. Preferably, the label binding substance and the capture binding substance are antibodies that are each specific for β(1-3)-glucan of Candida albicans.

The invention herein also includes an assay for detecting for the presence of a fungus in a patient sample, comprising utilizing an antibody in an immunochromatographic lateral flow assay system that reacts with a deep-seated β(1-3)-glucan fungal antigen in an extract from the patient sample, and a kit for rapidly detecting Candida albicans in a patient swab sample that includes the above device a sample collection device.

### The Product

The assays of the invention herein rely on applying a specimen containing soluble or highly dispersed fungal antigen obtained from the sample onto a matrix defining a flow path including at least a labeling and a capture zone.

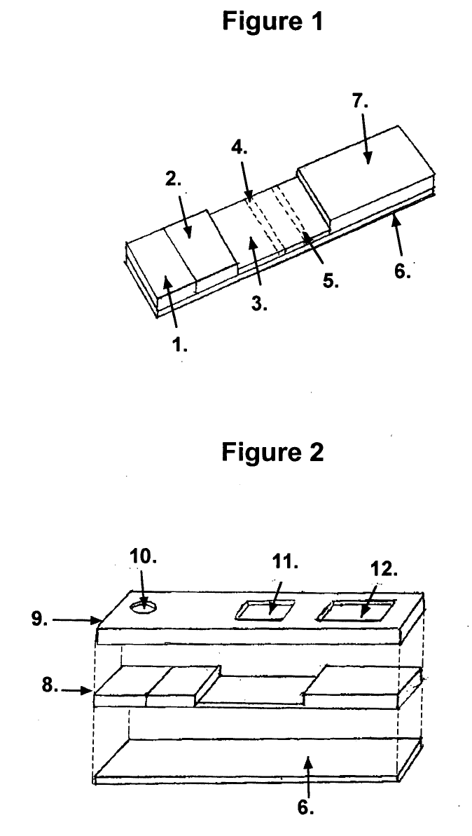

In one embodiment of the invention the first part of the matrix may be composed of a nonbibulous material, the sample-receiving pad 1 (FIG. 1), such as non-woven polyester. This pad 1 may be treated with various agents such as serum albumin in order to reduce any unspecific binding of the antigen. The sample volume is absorbed by the sample receiving pad 1 and moves by capillary forces into the labelling pad 2. This area is composed of non-woven polyester. The pad has been pre-absorbed with beads coated with monoclonal antibodies (A10A, dark blue) against β (1-3)-glucans or glucose oxidase (control, blue) by freezing and lyophilization. Any β (1-3)-glucan will bind to the dark blue beads via A10A to form complexes and move downstream into the immobilization pad 3. In parallel, glucose oxidase coated beads are carried by the aqueous flow into this area. The β (1-3)-glucan complexes are immobilized in the immobilization zone 4 by antibodies directed against the very same epitope on the β (1-3)-glucan (repetitive meres of glucose residues) (or another antigen epitope(s), i.e. reacting with B3B, or polyclonal antibody). This capture antibody is bound to the matrix material and thus fixed in a thin line across the pad. The control beads coated with glucose oxidase are immobilized further downstream by polyclonal antibodies against glucose oxidase bound to the pad in a thin line in the control zone 5. In the control zone 5, a blue stained band should always appear as a sign for a correct run. A dark blue band should turn up in front of the blue band when a positive sample is analyzed.

The device is preferably composed of a back 6 containing the various pads, together the assay matrix 8 in FIG. 2, and a plastic cover 9 arranged according to the drawing in FIG. 2. The sample to be analyzed is applied to the sample application port 10 and allowed to spread into the sample receiving pad 1. The blue/dark blue stained bands can be observed in the viewing window 11. An air outlet 12 is located at the end of the flow path so that the applied sample is flowing evenly through the various areas of the device.

The features of the present invention will be more clearly understood by reference to the following examples, which are not to be construed as limiting the invention.

### EXAMPLE 1

### Preparation of Yeast Cells, Cell Wall Fragments and Glucans.

Fungal growth. Candida albicans was grown in BHI medium over night (18 h) at 37° C. The microorganisms were washed three times by centrifugation at 1200 g for 10 min using PBS as washing fluid. After the last centrifugation, the supernatant was removed and the yeast cells diluted by distilled water to 10⁹ cells per ml, aliquotted, and frozen at −70° C.

Cell wall fragments. C. albicans (CaCW) cell wall fragments were prepared by agitation of the yeast cells with glass beads (Kondori, 2003 #32). The glucan structure of CaCW is composed of β(1-3) and β(1-6) glucans.

Carbohydrates from fungal cell wall, glucan. Glucan from Saccharomyces cerevisiae with β(1-6)-branched β(1-3)-linked glucose residues [β(1-3)(1-6)] was purchased from Sigma (St. Louis, Mo. USA). This glucan was dissolved in 0.3 M NaOH at a concentration of 20 mg/ml.

### EXAMPLE 2

### Preparation of Antibody to Fungal Cell Wall Component

The murine monoclonal antibodies used in the examples herein are of the IgM isotype (A10A, and B3B) and directed against β(1-3)(1-6)D-glucan, a cell wall component present in all medical important fungi. The antibodies were purified by size separation chromatography and dialyzed against saline. NaN₃ was added as a preservative and the concentration adjusted to 10 mg/ml. Its immunoreactivity was confirmed by analysis with ELISA using C. albicans cell wall fragments, and purified β(1-3)(1-6) glucan as coating antigens. Antibodies A10A and B3B were deposited on Mar. 20, 2007, under the Budapest Treaty, as DSM Numbers ACC2827 and ACC2828 respectively, at the DSMZ-Deutsche Sammlung von Microorganismen und Zellkulturen GmbH depository (Braunschweig, Germany). All restrictions upon availability to the public of these antibodies will be irrevocably removed upon granting of the patent.

### EXAMPLE 3

### Preparation of the Sample Receiving Pad

The sample receiving pad of the invention is prepared from nonwoven polyester. It is rendered nonadsorbent by incubating the pad in a solution containing 1% bovine serum albumin (BSA) in 50 mM TRIS-HCL buffer, pH 8.0. The pad is then incubated for 30 min at room temperature. The pad is thereafter frozen and lyophilized.

### EXAMPLE 4

### Preparation of the Labeling Pad

Test zone. Dark-blue-coloured beads were prepared at 1.25% solids with 25 mM TRIS-HCL buffer, pH 8.0, containing 0.5 mg/ml of A10A, final concentration, and rotated over night at room temperature. The beads were centrifuged once and the supernatant removed by aspiration. The beads were resuspended in 0.5 ml of 1% bovine serum albumin (BSA) in 50 mM TRIS-HCL buffer, pH 8.0, containing 1% sucrose. The mixture was rotated end-over-end for 4 h at room temperature. The bead preparation was centrifuged, the supernatant removed and the pellet resuspended in 1% BSA at 1% solids.

Control zone. A control zone was prepared by using blue beads and glucose oxidase at a concentration of 0.5 mg/ml. A similar procedure as for antibody labeling was performed.

For preparing the labeling zone the two bead solutions were diluted with 1% BSA in 50 mM TRIS-HCL buffer, pH 8.0, diluted to 0.04%. The mixture was poured onto the absorbing pad (nonwoven polyester) at 40 μl/cm² and lyophilized after freezing.

### EXAMPLE 5

### Preparation of the Immobilization Pad

The antibody used for immobilizing the labeled sample antigen can be either the same antibody as above, A10A or B3B (binding to another epitope on the glucan molecule) or may be any portion thereof (e.g., IgG-like, Fab or F(ab)₂ antigen binding fragments derived from the IgM antibody as known in the art) or any other appropriately reactive antibody that binds to the antigen as required in the invention herein. To prepare the immobilization pad, the antibody is diluted in PBS (1 mg/ml) and the solution applied to the 10 mm long pad (nitrocellulose membrane) as a thin line. The thin bands, making up the immobilization zone are then allowed to dry. The control polyclonal antibody solution (anti-glucose oxidase, 2 mg/ml) is then added in a similar thin line and allowed to dry. The anti-glucan and control antibody bands were placed 3 mm apart on the pad. The pad is thereafter treated with 1% BSA in TRIS-HCL (pH 8.0) and incubated for 1 h at room temperature. The pad is blotted and air-dried, and thereafter stored in a desiccator until assembled. The width of the pad is 15 mm.

### EXAMPLE 6

### Device Assembly

To assemble the device of the invention, a 20×9 mm strip of the immobilization pad 3 is affixed centrally on an adhesive transparency strip (700×17 mm) (not shown) on the back 6 made of double-sided adhesive tape (3M, St. Paul, Minn., USA). The labeling pad 2 is then affixed onto the back 6 with the adhesive strip next to the immobilization pad 3 with 1 mm overlap (not shown). Sample receiving pad 1 is then placed next to the labeling pad 2 with 1 mm overlap (not shown). The absorbent pad 7 is affixed to the distal end of the immobilization pad 3 with a 1 mm overlap (not shown). The assay matrix 8 on the on the back 6 is covered with a plastic cover 9 (FIG. 2). The immobilization lines are exposed in the viewing window 11. The sample application port 10 is placed above the sampling receiving pad 1.

### EXAMPLE 7

### Sample Collection Device for Vaginal Yeast Detection

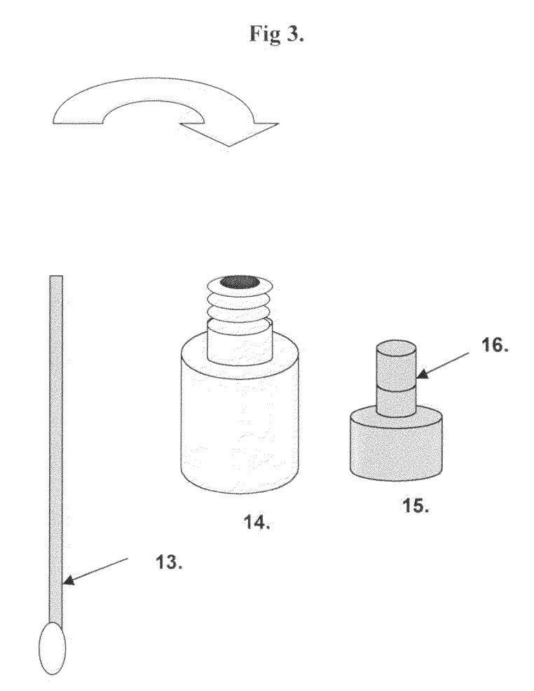

To use the invention for detection of vaginal yeast, a vaginal sample is collected by sample collection device as known in the art such as a cotton swab 13 (FIG. 3). The cotton swab 3 is submerged into a bottle 14 containing 1 ml of a TRIS-HCL buffer (20 mM, pH 7.2), 50 mM NaCl, 1% serum albumin, and 1% sucrose. The cotton swab 13 is rotated in the solution for 10 seconds. Thereafter it is removed from the bottle. A special cap 15 is put on tightly and the bottle 14 is shaken for some seconds. The top 16 of the cap 15 is snapped off and the bottle 14 turned upside down, and the solution from the bottle 14 is added to the sample receiving port 10 of the test device of the invention.

While the invention has been described with reference to specific embodiments, it will be appreciated that numerous variations, modifications, and embodiments are possible, and accordingly, all such variations, modifications, and embodiments are to be regarded as being within the spirit and scope of the invention.

I claim:

1. A device for rapid detection of fungi in a sample suspected of containing fungi, comprising:
a) an assay matrix defining a matrix flow path, said matrix flow path comprising a labeling pad and an immobilization pad;
b) a labeling complex present within the labeling pad that comprises a visible label bound to a labeled binding substance specific for a fungal β(1-3)-glucan antigen; and
c) a capture binding substance immobilized within the immobilization pad that is specific for the fungal β(1-3)-glucan antigen;
wherein the presence of the fungus is detected by the presence of the fungal antigen.

2. The device of claim 1, wherein the fungal antigen is from Candida albicans.

3. The device of claim 1, wherein the sample is selected from the group consisting of a sample from a patient, a food sample, and a feed sample.

4. The device of claim 1, wherein the sample is a vaginal swab sample from a patient.

5. The device of claim 1, wherein the label binding substance and the capture binding substance are antibodies that are each specific for β(1-3)-glucan of Candida albicans.

6. The device of claim 1, wherein the labeled binding substance and the capture binding substance are selected from the group consisting of the monoclonal antibodies, A1OA and B3B, which are reactive to β(1-3)-glucan.

7. A device for rapid detection of fungi in a sample suspected of containing fungi, comprising:
a) an assay matrix, having a proximal end and a distal end, comprising a piece of double-sided adhesive tape having a back; an immobilization pad affixed to an adhesive transparency strip placed on the back of the double-sided adhesive tape, a labeling pad affixed onto the back of the double-sided adhesive tape at the proximal end of the immobilization pad, a sample receiving pad placed proximal to the labeling pad, and an absorbent pad affixed to the distal end of the immobilization pad;
b) a plastic cover covering the assay matrix, the plastic cover having a sample port over the sample receiving pad, a viewing window over the immobilization paid, and an air outlet over the absorbent paid, so that when sample is placed in the sample port opening, the sample is absorbed by the sample receiving pad and moves by capillary forces into the labeling pad, and immobilization lines formed on the immobilization pad are exposed in the viewing window;
c) a labeling complex present within the labeling pad that comprises a visible label bound to a labeled binding substance specific for a fungal β(1-3)-glucan antigen; and
d) a capture binding substance immobilized within the immobilization pad that is specific for the fungal β(1-3)-glucan antigen;
wherein the presence of the fungus is detected by the presence of the fungal antigen.

8. The device of claim 7, wherein the sample receiving pad is made of nonbibulous material.

9. The device of claim 7, wherein the sample receiving pad is treated with serum albumin to reduce unspecific binding of the antigen.

10. The device of claim 7, wherein the labeling pad is made of non-woven polyester, pre-absorbed with beads coated with monoclonal antibodies against β (1-3)-glucans and beads coated with glucose oxidase as a control.

11. The device of claim 7, wherein the sample is selected from the group consisting of a sample from a patient, a food sample, and a feed sample.

12. The device of claim 7, wherein the sample is a vaginal swab sample from a patient.

13. A method for rapid detection of fungi in a biological sample suspected of containing fungi, comprising:
a) obtaining the biological sample with a sample collection device;
b) removing the biological sample from the sample collection device into a sample suspension fluid; and
c) adding sample suspension fluid to a sample receiving port of the device of claim 1.

14. A method for rapid detection of fungi in a biological sample suspected of containing fungi, comprising:
a) obtaining the biological sample with a sample collection device;
b) removing the biological sample from the sample collection device into a sample suspension fluid; and
c) adding sample suspension fluid to a sample receiving port of the device of claim 7.

15. A bedside kit, comprising:
a) the device for rapid detection of fungi in a sample suspected of containing fungi according to claim 7; and
b) a sample collection device.
